(19) United States
(12) Patent Application Publication
(10) Pub. No.: US 2012/0245693 A1
(43) Pub. Date: Sep. 27, 2012

(54) SPINAL FIXATION DEVICE

(76) Inventors: Josef Gorek, Ross, CA (US); Scott Jones, McMurray, PA (US)

(21) Appl. No.: 13/430,084

(22) Filed: Mar. 26, 2012

Related U.S. Application Data
(60) Provisional application No. 61/467,481, filed on Mar. 25, 2011.

(51) Int. Cl. A61F 2/44

(52) U.S. Cl. 623/17.16

(57) ABSTRACT

A spinal fixation device includes a proximal surface having a first portion defining a first plane and a second portion defining a second plane. The first and second planes define a first angle in relation to each other. The first and second planes define first through holes and a second through hole, respectively. The first through holes each define a first longitudinal axis, and the second through hole defines a second longitudinal axis. In particular, the first and second longitudinal axes define a second angle in relation to each other. The first through holes define a first center line on the first plane, and the second through hole defines a second center line on the second plane. The first and second center lines are disposed on opposite sides of a center line defined by the proximal surface.

### CROSS REFERENCE TO RELATED APPLICATIONS

This application claims priority to, and the benefit of, U.S. Provisional Patent Application Ser. No. 61/467,481 filed Mar. 25, 2011, the entire contents of which are incorporated by reference herein.

### BACKGROUND

1. Technical Field

The present disclosure relates generally to orthopedic surgery with particular regard to spinal surgery. Specifically, the present disclosure relates to an apparatus and methods for fixating adjacent vertebral bodies relative to each other.

2. Description of Related Art

The spinal column is a complex system of bones and connective tissues that provide support for the human body and protection for the spinal cord and nerves. The adult spine is comprised of an upper and lower portion. The upper portion contains 24 discrete bones, which are subdivided into three areas including 7 cervical vertebrae, 12 thoracic vertebrae and 5 lumbar vertebrae. The lower portion is comprised of the sacral and coccygeal bones. The cylindrical shaped bones, called vertebral bodies, progressively increase in size from the upper portion downwards to the lower portion.

An intervertebral disc along with two posterior facet joints cushion and dampen the various translational and rotational forces exerted upon the spinal column. The intervertebral disc is a spacer located between two vertebral bodies. The facets provide stability to the posterior portion of adjacent vertebrae. The spinal cord is housed in the canal of the vertebral bodies. It is protected posteriorly by the lamina. The lamina is a curved surface with three main protrusions. Two transverse processes extend laterally from the lamina, while the spinous process extends posteriorly. The vertebral bodies and lamina are connected by a bone bridge called the pedicle.

The spine is a flexible structure capable of a large range of motion. There are various disorders, diseases and types of injury which restrict the range of motion of the spine or interfere with important elements of the nervous system. The problems include, but are not limited to, scoliosis, kyphosis, excessive lordosis, spondylolisthesis, slipped or ruptured discs, degenerative disc disease, vertebral body fracture, and tumors. Persons suffering from any of the above conditions typically experience extreme or debilitating pain and often times diminished nerve function.

Spinal fixation apparatuses are widely employed in surgical processes for correcting spinal injuries and diseases. When the disc has degenerated to the point of requiring removal, there are a variety of interbody implants that are utilized to take the place of the disc. These include, PEEK interbody spacers, metal cages and cadaver and human bone implants. In order to facilitate stabilizing the interbody, other implants are commonly employed, including longitudinally linked rods secured to coupling elements, which in turn are secured to the bone by spinal bone fixation fasteners such as pedicle screws, hooks, and others. The opposing pair of longitudinally linked rods is commonly disposed along the long axis of the posterior spine. In lieu of using rods, a plate and screw system can be utilized to secure the anterior or lateral portion of the spine. The plate provides added stability and a means for preventing expulsion of the interbody.

To meet the problem of preventing expulsion of the interbody device and for providing stability to the anatomy, a need exists for an spinal fixation device that can be secured to the spine and provide anterior column support and stabilization, while providing a maximum fusion area. The disclosed spinal fixation device may be implanted, in part, anteriorly of the anterior margins of the vertebral bodies. It is also contemplated that the disclosed spinal fixation device may have a configuration such that the most anterior portion of the spinal fixation device extends beyond the planar limits defined and/or demarcated by the planes of the vertebral endplates where the spinal fixation device is situated.

### SUMMARY

In accordance with an embodiment of the present disclosure, there is provided a spinal fixation device. The spinal fixation device includes a proximal surface having a first portion defining a first plane and a second portion defining a second plane. The first and second planes define a first angle in relation to each other. The first plane defines a plurality of first through holes, and the second plane defines a second through hole. In addition, the plurality of first through holes each defines a first longitudinal axis, and the second through hole defines a second longitudinal axis. In particular, the first and second longitudinal axes define a second angle in relation to each other. Moreover, the plurality of first through holes defines a first center line on the first plane, and the second through hole defines a second center line on the second plane. The first and second center lines are disposed on opposite sides of a center line defined by the proximal surface.

In an embodiment, the first angle may be an acute angle. In particular, the first angle may range between about 20 degrees and about 85 degrees. In an embodiment, the first angle may be about 75 degrees. The second angle may range between about 10 degrees and about 90 degrees. It is contemplated that the second angle may be about 85 degrees.

In another embodiment, the first through holes may be in an orthogonal relation with the first plane and the second through hole may be in an orthogonal relation with the second plane. At least one of the first through holes and the second through hole may be threaded.

In yet another embodiment, at least one of the first through holes and the second through hole may define a binding region formed from commercially pure titanium.

In accordance with another aspect of the present disclosure, there is provided a method of stabilizing a spine. The method includes providing a spinal fixation device including a body having a proximal surface having a first portion defining a first plane and a second portion defining a second plane. The first and second planes define an acute angle with respect to each other. The first plane defines a plurality of first through holes, and the second plane defines a second through hole. The method further includes distracting a disc space, preparing a fusion bed, inserting the spinal fixation device between vertebral bodies, and fixing the spinal fixation device to the vertebral bodies.

In an embodiment, the method may further include decompressing neural elements. In addition, the method may further include inserting an interbody device. The method may also include adjusting a gap between the interbody device and the spinal fixation device.

Inserting an interbody device may include placing the interbody device posteriorly on an apophyseal ring of the vertebral bodies.

In yet another embodiment, the method may also include holding the spinal fixation device in alignment with the interbody device during insertion into the disc space. In addition, inserting the spinal fixation device between vertebral bodies may include placing the spinal fixation device anteriorly on the vertebral bodies. Alternatively, the spinal fixation device may be positioned such that a portion of the spinal fixation device extends anteriorly of the anterior cortex of one of the vertebral bodies.

In still yet another embodiment, fixing the spinal fixation device to the vertebral bodies may include inserting bone anchors through respective through holes.

In still yet another embodiment, the plurality of first through holes may each define a first longitudinal axis, and the second through hole may define a second longitudinal axis. The first and the second longitudinal axes may define an acute angle in relation to each other. In addition, the plurality of first through holes may define a first center line, and the second through hole may define a second center line. The first and second center lines may be disposed on opposite sides of a center line defined by the proximal surface. In addition, the plurality of bone anchors may have different dimensions.

### BRIEF DESCRIPTION OF THE DRAWINGS

The above and other aspects and features of the present disclosure will become more apparent in light of the following detailed description when taken in conjunction with the accompanying drawings in which:

FIG. 1 is a perspective view of a spinal fixation device in accordance with an embodiment of the present disclosure illustrating use with bone anchors;

FIG. 2 is a front view of the spinal fixation device of FIG. 1 without the bone anchors;

FIG. 3 is a top view of the spinal fixation device of FIG. 2;

FIG. 4 is a front view of the spinal fixation device of FIG. 2;

FIG. 5 is a side view of the spinal fixation device of FIG. 2;

FIG. 6 is a side, cross-sectional view of the spinal fixation device of FIG. 2;

FIG. 7 is a side, cross-sectional view of the spinal fixation device of FIG. 1 in situ;

FIG. 8 is a front view of the spinal fixation device of FIG. 1 in situ; and

FIG. 9 is a side view of the spinal fixation device of FIG. 1 in situ.

### DETAILED DESCRIPTION OF THE EMBODIMENTS

Particular embodiments of the present disclosure will be described herein with reference to the accompanying drawings. As shown in the drawings and as described throughout the following description, and as is traditional when referring to relative positioning on an object, the terms “proximal” and “trailing” may be employed interchangeably, and should be understood as referring to the portion of a structure that is closer to a clinician during proper use. The terms “distal” and “leading” may also be employed interchangeably, and should be understood as referring to the portion of a structure that is farther from the clinician during proper use. In addition, the term “cephalad” is used in this application to indicate a direction toward a patient's head, whereas the term “caudad” indicates a direction toward the patient's feet. Further still, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front. In the following description, well-known functions or constructions are not described in detail to avoid obscuring the present disclosure in unnecessary detail.

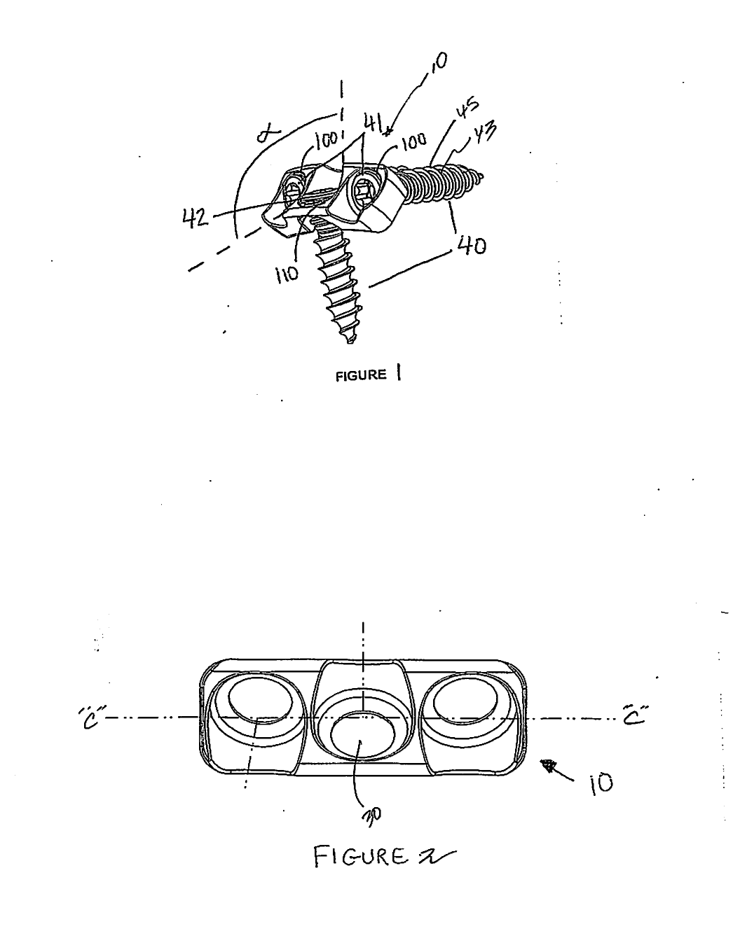

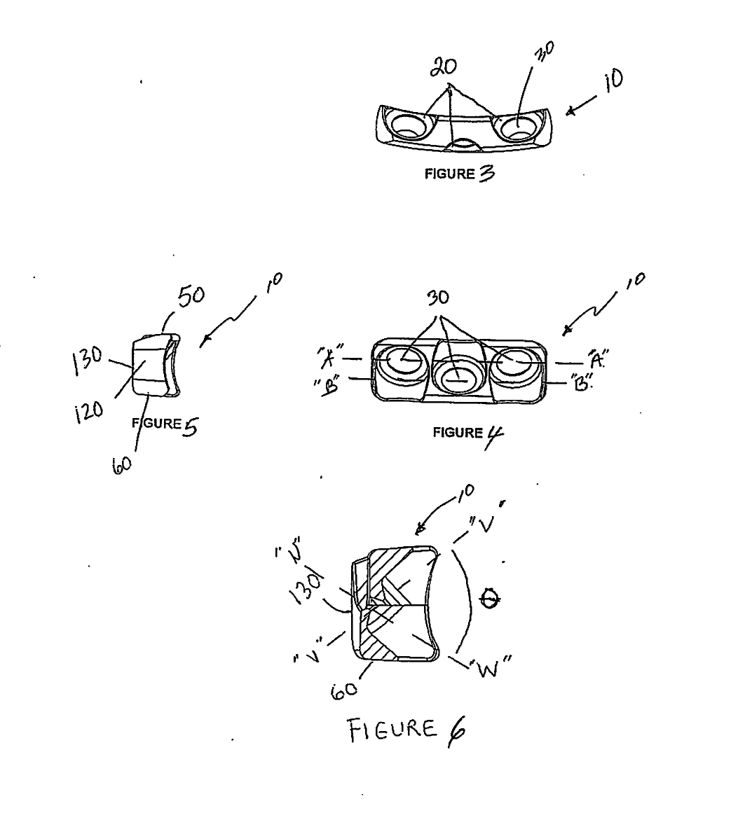

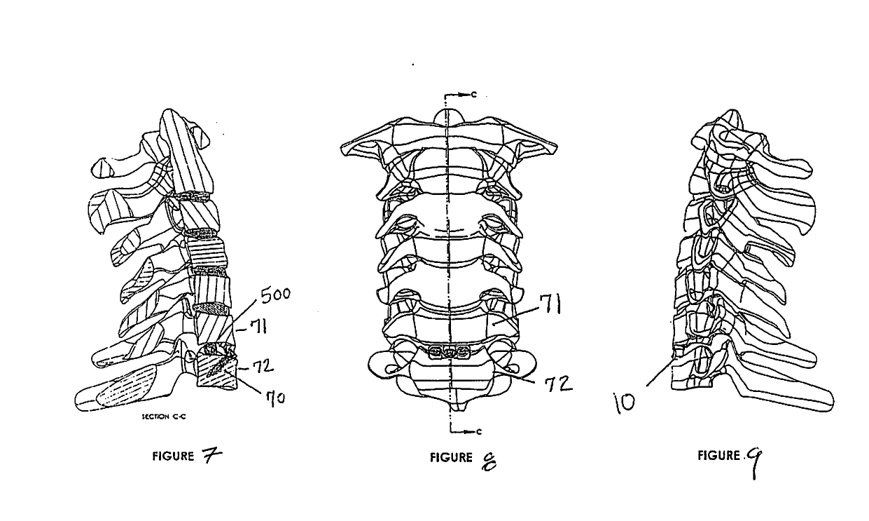

With reference to FIGS. 1, 2 and 5, a spinal fixation device in accordance with an embodiment of the present disclosure is shown and generally identified as 10. Spinal fixation device 10 includes a body having split proximal surfaces 100, 110 that define two adjacent planes, top and bottom surfaces 50, 60 extending distally from proximal surfaces 100, 110, a pair of side surfaces 120 extending between top and bottom surfaces 50, 60 and a distal surface 130. The body is configured for positioning at least partially within an intervertebral space between adjacent vertebrae. In particular, spinal fixation device 10 may sit extend beyond of an anterior rim of a vertebral body by about 1 to 2 mm and may serve as a partial intradiscal device, i.e., spinal fixation device 10 need not be disposed entirely between the vertebral bodies. It is contemplated that the disclosed spinal fixation device 10 may be positioned between adjacent vertebral bodies such that a portion of the spinal fixation device 10 extends about 1 to 2 mm anteriorly of the anterior cortex of one of the vertebral bodies. Alternatively, a portion of the spinal fixation device 10 may extend in the cephalad or caudad direction beyond the limits defined by the planes of the endplates of the adjacent vertebral bodies. Spinal fixation device 10 may be used in conjunction with an interbody device 500 (FIG. 7), as will be described hereinbelow. By maintaining separation of interbody device 500 (cage or graft) and spinal fixation device 10, the optimal position of each device within the disc space may be achieved, since each device 10, 500 is movable independent of the other, which in turn maximizes and optimizes the load sharing characteristics of both. In particular, optimal placement of spinal fixation device 10 enables better screw fixation (e.g., fixation at the anterior rim of the vertebral body) and better screw angle trajectory. Moreover, spinal fixation device 10 keeps interbody device 500 in position between adjacent vertebral bodies and inhibits expulsion of interbody device 500 from the intervertebral space. In addition, a biologic material may be placed between a gap between spinal fixation device 10 and interbody device 500.

With particular reference to FIG. 1, proximal surfaces 100, 110 are disposed at an angle α with respect to each other. Angle α may range between about 20° and about 85°. In an embodiment, the angle α is about 75°. The body defines a plurality of through holes 30 configured and dimensioned to receive bone anchors 40 therethrough. First through holes 30 extend through proximal surface 100 at an orthogonal relation therewith, and a second through hole 30 extends through proximal surface 110 at an orthogonal relation therewith. Under such a configuration, a pair of bone anchors 40 are inserted into, for example, cephalad vertebra 71 (FIG. 7), and a single bone anchor 40 is inserted into a caudad vertebra 72 (FIG. 7).

Bone anchor 40 includes a shaft 43 and a head 41, 42 attached thereto. Shaft 43 includes helical threads 45 peripherally disposed around an outer surface thereof. Threads 45 may be adapted for threadably mating with cortical bone or with cancellous bone. Bone anchor 40 may be formed from any suitable biocompatible material such as titanium, titanium alloys, PEEK, or stainless steel. It is contemplated that all or a portion of bone anchor 40 may be formed from a resorbable material, as is known in the art. Further still, bone anchor 40 may be formed of several materials including metallic and polymeric materials.

With reference to FIG. 3, each through hole 30 includes a binding region 20. Binding region 20 is formed from commercially pure titanium. Threads 45 on an exterior of head 41, 42 of bone anchors 40 are formed from a titanium alloy such as Ti-6Al-4V, which is harder than the commercially pure titanium of binding region 20. Under such a configuration, when bone anchor 40 threadably engages binding region 20, binding region 20 is deformed and inhibits bone anchor 40 from backing out of and separating from binding region 20. An example of this locking arrangement is disclosed in U.S. Pat. No. 6,322,562 to Wolter, the entire contents of which are hereby incorporated by reference. Alternatively, through hole 30 may include complementary threads for forming a secure attachment with bone anchors 40.

Furthermore, binding regions 20 are disposed to provide the farthest distance between the cranial and caudal heads 41, 42 of bone anchor 40, while maintaining the desired mechanical interconnection strength of spinal fixation device 10. Such a configuration provides an optimized screw trajectory 70 in relation to the vertebral endplates and vertebral bodies as well as in relation to interbody device 500 (allograft, cage etc) (FIG. 7). In addition, the need to use a specially shaped spacer or graft is eliminated or minimized. Such arrangement of spinal fixation device 10 minimizes mass and volume of metal within the disc space to allow for maximum space for interbody device (the graft and/or cage) 500, and thus increasing the likelihood of achieving fusion. Separately placing interbody device 500 (cage or graft) and spinal fixation device 10 effects optimal placement of spinal fixation device 10, which improved screw fixation, screw angle trajectory and the ability to use the anterior rim of the vertebral body for screw fixation, as well as maximizing and optimizing the load sharing characteristics of both components 10, 500.

With reference now to FIGS. 4 and 6, through holes 30 defined in proximal surface 100 define a centerline “A-A” and through hole 30 defined in proximal surface 110 defines a centerline “B-B.” Centerlines “A-A,” “B-B” are defined on opposite sides of a centerline “C-C” (FIG. 2) defined by spinal fixation device 10. In addition, each through hole 30 defined in proximal surface 100 defines a longitudinal axis “V-V” and through hole 30 defined in proximal surface 110 defines a longitudinal axis “W-W.” Longitudinal axes “V-V,” “W-W” define an angle Θ ranging between about 10° and about 90°. In particular, in an embodiment angle Θ may be about 85°. At this angle, the maximum distance between bone anchor trajectories to provide optimal purchase in the anatomy is achieved, as well as a minimally sized implant that allows for maximum bone graft in the disc space.

Proximal surfaces 100,110 are disposed exteriorly of the vertebral space to permit insertion of bone anchor 40 into through holes 30 and into the adjacent vertebrae. Bone anchor 40 extends in a substantially perpendicular orientation relative to the surface from which it extends. The device 10 can be secured to the vertebra above and below the disc space utilizing through holes 30 and bone anchors 40 to provide stability to the spinal segment and to diminish the likelihood of interbody cage or graft 500 expulsion.

With reference now to FIGS. 7-9, in use, spinal fixation device 10 can be used in a standard anterior cervical discectomy and fusion (ACDF) procedure, where a discectomy is performed coupled with distraction of the disc space, decompression of any neural elements, and preparation of the fusion bed followed by insertion of an interbody device 500 and a spinal plate (not shown). Instead of putting a spinal plate onto the front (anterior) portion of the vertebral bodies, interbody 500 (graft, cage etc) is placed posteriorly on the apophyseal ring of the vertebral bodies and spinal fixation device 10 is placed anteriorly on the vertebral bodies. Upon accurate placement of spinal fixation device 10, as well as interbody device 500, bone anchors 40 can be inserted through holes 30 of spinal fixation device 10 to fix its location. Bone anchors 40 can be of various diameters and lengths to allow for a customized fixation. For example, one larger diameter bone anchor 40 may be used in through hole 30 defined in proximal surface 110, while two smaller diameter bone anchors 40 may be used in proximal surface 100 to provide the same fixation in the cranial and caudal vertebrae 71, 72.

In addition, various instruments such as, e.g., a holding device (not shown), may be utilized to hold spinal fixation device 10 in alignment with interbody device 500 (cage or spacer) during insertion into the disc space and also during bone anchor 40 placement. In addition, the holding device may be utilized to adjust a gap between spinal fixation device 10 and interbody device 500. Furthermore, the holding device may be utilized to deliver graft between spinal fixation device 10 and interbody device 500. For instance demineralized bone graft putty, which is malleable, could be passed through one or more of the screw holes before or after fixation with one or two of the bone screw anchors. In particular, the holding device may include tines that are configured and dimensioned for passage through holes 30 of spinal fixation device 10 and secure engagement with interbody device 500. The instruments are subsequently removed from the surgical site, leaving two independent devices (spinal fixation device 10 and interbody device 500) in the disc space which can be further manipulated independently. The holding device can also set the distance or gap between interbody device 500 and spinal fixation device 10 should it be desired. This might be accomplished by a threaded piston on the holding device that could project through the center screw hole 30 of the spinal fixation device 10 and abut the anterior surface of the graft or interbody device 500. Alternatively, the tines of the holding device might have a “shoulder, step” that could project through the screw holes of the spinal fixation device 10 and abut the anterior surface of the graft or interbody device 500.

Spinal fixation device 10 may be manufactured from various biocompatible materials including, PEEK, titanium, and stainless steel. However, it is contemplated that spinal fixation device 10 may be made of metal and not PEEK (polyetheretherketone) to facilitate the fusion process. Interbody device 500 need not be constructed from PEEK or metal and may be formed from another polymeric material or bone. Spinal fixation device 10 may be used in combination with an allograft interbody device 500 which is optimal for structural support and fusion. Interbody devices 500 utilizing PEEK requires a larger footprint than metal to achieve the same structural integrity, and does not promote fusion.

The present disclosure provides an improved spinal fixation system. One skilled in the art will recognize that the disclosure is not limited to use in the cervical region or spine surgery, and that the instrument and methods can be adapted for use with any suitable surgical device. For example, it is understood by one of ordinary skill in the art that spinal fixation device 10 may vary in shape, length, width and height, as well as the number of through holes 30 defined therein. Those skilled in the art will appreciate that the present disclosure may be implemented in a number of different applications and embodiments and is not specifically limited in its application to the particular embodiments depicted herein.

What is claimed is:

1. A spinal fixation device configured for positioning at least partially within an intervertebral space between adjacent vertebrae, the spinal fixation device comprising:
a proximal surface having a first portion defining a first plane and a second portion defining a second plane, the first and second planes defining a first angle in relation to each other, the first plane defining a plurality of first through holes, the second plane defining a second through hole, the plurality of first through holes each define a first longitudinal axis, the second through hole defines a second longitudinal axis, the first and the second longitudinal axes define a second angle in relation to each other, the plurality of first through holes define a first center line on the first plane, and the second through hole defines a second center line on the second plane, the first and second center lines disposed on opposite sides of a center line defined by the proximal surface.

2. The spinal fixation device according to claim 1, wherein the first angle is an acute angle.

3. The spinal fixation device according to claim 2, wherein the first angle ranges between about 20 degrees and about 85 degrees.

4. The spinal fixation device according to claim 2, wherein the first angle is about 75 degrees.

5. The spinal fixation device according to claim 1, wherein the first through holes are in an orthogonal relation with the first plane and the second through hole is in an orthogonal relation with the second plane.

6. The spinal fixation device according to claim 1, wherein at least one of the first through holes and the second through hole is threaded.

7. The spinal fixation device according to claim 1, wherein at least one of the first through holes and the second through hole defines a binding region formed from commercially pure titanium.

8. The spinal fixation device according to claim 1, wherein the second angle ranges between about 10 degrees and about 90 degrees.

9. The spinal fixation device according to claim 8, wherein the second angle is about 85 degrees.

10. A method of stabilizing a spine, the method comprising:
providing a spinal fixation device including:
a body having a proximal surface having a first portion defining a first plane and a second portion defining a second plane, the first and second planes defining an acute angle with respect to each other, the first plane defining a plurality of first through holes, the second plane defining a second through hole;
distracting a disc space;
preparing a fusion bed;
inserting the spinal fixation device between vertebral bodies; and
fixing the spinal fixation device to the vertebral bodies.

11. The method of stabilizing a spine according to claim 10, further comprising decompressing neural elements.

12. The method of stabilizing a spine according to claim 10, further comprising inserting an interbody device.

13. The method of stabilizing a spine according to claim 12, wherein inserting an interbody device includes placing the interbody device posteriorly on an apophyseal ring of the vertebral bodies.

14. The method of stabilizing a spine according to claim 12, further comprising holding the spinal fixation device in alignment with the interbody device during insertion into the disc space.

15. The method of stabilizing a spine according to claim 10, wherein inserting the spinal fixation device between vertebral bodies includes placing the spinal fixation device anteriorly of the anterior cortex of at least one vertebral body.

16. The method of stabilizing a spine according to claim 10, wherein fixing the spinal fixation device to the vertebral bodies includes inserting bone anchors through respective through holes.

17. The method of stabilizing a spine according to claim 16, wherein the plurality of bone anchors have different dimensions.

18. The method of stabilizing a spine according to claim 12, further comprising adjusting a gap between the interbody device and the spinal fixation device.

19. The method of stabilizing a spine according to claim 10, wherein the plurality of first through holes each define a first longitudinal axis, and the second through hole defines a second longitudinal axis, the first and the second longitudinal axes defining an acute angle in relation to each other.

20. The method of stabilizing a spine according to claim 10, wherein the plurality of first through holes define a first center line on the first plane, and the second through hole defines a second center line on the second plane, the first and second center lines disposed on opposite sides of a center line defined by the proximal surface.
